(19) United States
(12) Patent Application Publication
(10) Pub. No.: US 2025/0046422 A1
(43) Pub. Date: Feb. 6, 2025

(54) METHOD AND SYSTEM FOR GENERATING GUIDE FOR DENTAL TREATMENT

(71) Applicant: Jeong Lan KIM, Seoul (KR)

(72) Inventor: Jeong Lan KIM, Seoul (KR)

(21) Appl. No.: 18/794,461

(22) Filed: Aug. 5, 2024

(30) Foreign Application Priority Data
Aug. 4, 2023 (KR) 10-2023-0102193

(51) Int. Cl. G16H 20/40; G06V 10/25; G06V 10/26; G06V 10/70; G06V 10/764; G06V 10/774; G06V 20/50; G16H 30/40

(52) U.S. Cl. CPC G16H 20/40; G06V 10/25; G06V 10/26; G06V 10/764; G06V 10/774; G06V 10/87; G06V 20/50; G16H 30/40; G06V 2201/03

(57) ABSTRACT

A method for generating a guide for dental treatment is executed by at least one processor and includes receiving, from a user terminal, a position code for a tooth of a plurality of teeth assigned different position codes and a dental image including the tooth, identifying a nerve region of the tooth in the received dental image, in which the nerve region of the tooth includes the tooth and a nerve tissue around the tooth, and outputting guide data for the tooth corresponding to the nerve region based on the position code and the identified nerve region.

### CROSS-REFERENCE TO RELATED APPLICATION

This application claims priority to Korean Patent Application No. 10-2023-0102193 filed on Aug. 4, 2023, the entire contents of which are herein incorporated by reference.

### Technical Field

The present disclosure relates to a method and a system for generating a guide for dental treatment, and specifically, to a method and a system for generating guide data for dental treatment such as an implant and nerve treatment based on a position code and dental images of a tooth and providing the generated guide data.

### Description of Related Art

The biggest cause of tooth loss is periodontal disease, which is an inflammation of the gums (periodental) that wrap and support the teeth, gum bones (alveolar bones), periodontal ligaments, etc., and can be caused by wrong brushing habits. If a tooth is lost or extraction is inevitable due to severe periodontal disease, quick treatment is needed, and if a missing tooth is left untreated, the balance between teeth will collapse, which will naturally cause teeth to move, slurred pronunciation, or even poor chewing and gastrointestinal disorders. For the treatment of periodontal diseases, implant treatment can be considered, which implants an artificial tooth in the gum bone. The implant is a treatment that cuts the gum, connects the abutment and the crown, and implants artificial root in the gum bone so as to restore the chewing and aesthetic functions similar to natural teeth, and it can restore up to 80% of the chewing function of natural teeth and can be used semi-permanently. Meanwhile, for a stable dental implant procedure, it is necessary to develop a systematic treatment plan that considers functional parts such as gum bone condition, oral structure, etc. that differ from individual to individual, because in order to increase satisfaction after surgery without side effects, it requires to accurately determine the overall oral structure of the patient and place the artificial root at an appropriate angle and depth.

Meanwhile, the root canal treatment, known as nerve treatment, is a procedure that removes damaged tissues when the endodontic is damaged due to gingivitis, periodontal diseases, etc., and fills a special material to restore the tooth so that the tooth naturally functions without pain. This treatment involves observing the patient's systemic condition, medical history, and dental conditions such as fractures and caries, and performing tests such as radiographic examinations, palpation and thermography, and cavity preparation examinations to diagnose the inflammatory condition of the pulp and apicectomy disease for root canal therapy. In the “access cavity preparation” process, which can be seen as the most important process of the root canal treatment, a doctor himself analyzes the CT image or uses a dental microscope to determine the shape of the tooth and the inner neural canal, and then prep it to create a passage for accessing the tooth, but there is a problem in that it is difficult to accurately identify the neural canal with this method, and excessive prep to find the neural canal can lead to unnecessary tooth removal, which can cause secondary damage to the tooth.

### SUMMARY

In order to solve one or more problems (e.g., the problems described above and/or other problems not explicitly described herein), the present disclosure provides a method and a system for generating a guide for dental treatment.

The present disclosure provides a method for generating a guide for dental treatment, which is capable of providing auxiliary indicators for the treatment by providing a guide for implant and/or nerve treatment using a machine learning model associated with a position code for a tooth in dental treatment such as implant and nerve treatment, a computer program stored in a recording medium, and a system (apparatus) including the same.

The present disclosure may be implemented in a variety of ways, including a method, a system (apparatus), or a computer program stored in a readable storage medium.

According to an embodiment of the present disclosure, a method for generating a guide for dental treatment may include receiving, from a user terminal, a position code for a tooth of a plurality of teeth assigned different position codes and a dental image including the tooth, identifying a nerve region of the tooth in the received dental image, in which the nerve region of the tooth may include the tooth and a nerve tissue around the tooth, and outputting guide data for the tooth corresponding to the nerve region based on the position code and the identified nerve region.

According to an embodiment, The identifying the nerve region of the tooth in the received dental image may further include selecting, from among a plurality of first machine learning models, a first machine learning model associated with the position code of the tooth, and inputting the nerve region of the tooth to the selected first machine learning model to perform segmentation on the nerve region.

According to an embodiment, the outputting the guide data may further include receiving, from the user terminal, a request to output first guide data, selecting, from among a plurality of second machine learning models, a second machine learning model associated with the position code of the tooth, and inputting the segmentation performed on the nerve region to the selected second machine learning model to output the first guide data.

According to an embodiment, the first guide data may include at least one of a distance between a root tip and a bone level in the nerve region, a thickness and a length of a periodontal ligament in the nerve region, or an amount of bone in the nerve region, and the first guide data may be overlaid on the dental image and output, and may be guide data for implant treatment.

According to an embodiment, the outputting the guide data may include receiving, from the user terminal, a request to output second guide data, selecting, from among a plurality of third machine learning models, a third machine learning model associated with the position code of the tooth, and inputting the segmentation performed on the nerve region to the selected third machine learning model to output the second guide data.

According to an embodiment, the second guide data may include at least one of prep guide data, coordinates of a neural canal opening in the tooth, a number of neural canals, a length of a neural canal, or a shape of the neural canal, and the second guide data may be overlaid on the dental image and output, and may be guide data for nerve treatment.

According to an embodiment, the method may further include classifying the plurality of teeth assigned the different position codes into at least one structure of central incisor, lateral incisor, canine, bicuspid, and molar, training the plurality of first machine learning models to perform the segmentation of the nerve region in accordance with the classified tooth structure, training the plurality of second machine learning models to output first guide data in accordance with the classified tooth structure, and training the plurality of third machine learning models to output second guide data in accordance with the classified tooth structure . . .

According to an embodiment, the identifying the nerve region of the tooth may include detecting a plurality of tooth regions from the dental image, and identifying the nerve region of the tooth based on the detected plurality of tooth regions and the received position code for the at least one tooth.

According to an embodiment of the present disclosure, a computer program stored in a computer-readable recording medium may be provided to execute the method for generating the guide for dental treatment on a computer.

According to an embodiment of the present disclosure, an information processing device is provided. The information processing device may include a memory, and at least one processor connected to the memory and configured to execute at least one computer-readable program included in the memory, in which the at least one program may include instructions for receiving, from a user terminal, a position code for at least one tooth of a plurality of teeth assigned different position codes and a dental image including the tooth, identifying a nerve region of the tooth in the received dental image, in which the nerve region of the tooth includes the tooth and a nerve tissue around the tooth, and outputting guide data for the tooth corresponding to the nerve region based on the position code and the identified nerve region.

According to some aspects of the present disclosure, by inputting the position code of the tooth and the nerve region of the tooth to the first machine learning model and performing segmentation on the nerve region, identification of the nerve region for providing the guide can be efficiently performed.

According to some aspects of the present disclosure, by inputting the position code and the segmentation of the tooth to the second machine learning model and/or the third machine learning model to generate the guide data for the tooth, it is possible to provide a guide for objective and accurate implant and/or nerve treatment.

According to some aspects of the present disclosure, by identifying the nerve region of the tooth from the dental image and generating the guide data based on the same, regions in the image unnecessary for the implant or nerve treatment can be removed, thereby significantly improving processing speed and accuracy.

According to some aspects of the present disclosure, by detecting the tooth region from the dental image and identifying the nerve region of the tooth based on the same, the accuracy of identification of nerve region of the teeth can be improved, and auxiliary indicators for more accurate diagnosis can be provided.

According to some aspects of the present disclosure, by selecting a machine learning model suitable for the position code of the tooth, more suitable guide data can be generated in accordance with the structure of the tooth to be treated.

According to some aspects of the present disclosure, by generating the guide data for implant treatment such as the distance between the root tip and the bone level, the thickness and the length of the periodontal ligament, the amount of bone, etc., more accurate implant treatment can be performed.

According to some aspects of the present disclosure, by generating the guide data for nerve treatment such as the prep guide data, the coordinates of the neural canal opening in the tooth, the number of neural canals, the length of the neural canal, the shape of the neural canal, etc., more accurate nerve treatment can be performed.

The effects of the present disclosure are not limited to the effects described above, and other effects not mentioned will be able to be clearly understood by those of ordinary skill in the art (referred to as “those skilled in the art”) from the description of the claims.

### BRIEF DESCRIPTION OF THE DRAWINGS

Aspects of the present disclosure will be described with reference to the accompanying drawings described below, where similar reference numerals indicate similar elements, but is not limited thereto, in which:

FIG. 1 is a diagram illustrating an example of providing guide data according to an embodiment of the present disclosure;

FIG. 2 is a block diagram illustrating an internal configuration of a user terminal and an information processing system according to an embodiment of the present disclosure;

FIG. 3 is a diagram illustrating a position code of each of a plurality of teeth according to an embodiment of the present disclosure;

FIG. 4 is a diagram illustrating a nerve region of a tooth according to an embodiment of the present disclosure;

FIG. 5 is a block diagram illustrating a detailed configuration of a processor according to an embodiment of the present disclosure;

FIG. 6 is a flowchart illustrating a method for generating a guide for dental treatment according to an embodiment of the present disclosure;

FIG. 7 illustrates an example of a dental image according to an embodiment of the present disclosure;

FIG. 8 is a diagram illustrating an example in which segmentation is performed according to an embodiment of the present disclosure;

FIG. 9 is a diagram illustrating an example in which segmentation is performed according to another aspect according to an embodiment of the present disclosure;

FIG. 10 is a diagram illustrating an example of inputting a position code and a dental image to a machine learning model to generate segmentation data according to an embodiment of the present disclosure;

FIG. 11 is a diagram illustrating an example of implant guide data according to an embodiment of the present disclosure;

FIG. 12 is a diagram illustrating an example of implant guide data according to another embodiment of the present disclosure;

FIG. 13 is a diagram illustrating an example of nerve treatment guide data according to an embodiment of the present disclosure;

FIG. 14 is a diagram illustrating an example of inputting a position code and segmentation to generate first guide data according to an embodiment of the present disclosure;

FIG. 15 is a diagram illustrating an example of inputting a position code and segmentation to generate second guide data according to an embodiment of the present disclosure; and

FIG. 16 illustrates an example of an artificial neural network model according to an embodiment of the present disclosure.

### DETAILED DESCRIPTION

Hereinafter, example details for the practice of the present disclosure will be described in detail with reference to the accompanying drawings. However, in the following description, detailed descriptions of well-known functions or configurations will be omitted when it may make the subject matter of the present disclosure rather unclear.

In the accompanying drawings, the same or corresponding components are assigned the same reference numerals. In addition, in the following description of various examples, duplicate descriptions of the same or corresponding components may be omitted. However, even if descriptions of components are omitted, it is not intended that such components are not included in any example.

Advantages and features of the disclosed examples and methods of accomplishing the same will be apparent by referring to examples described below in connection with the accompanying drawings. However, the present disclosure is not limited to the embodiments disclosed below, and may be implemented in various forms different from each other, and the embodiments are merely provided to make the present disclosure complete, and to fully disclose the scope of the disclosure to those skilled in the art to which the present disclosure pertains.

The terms used herein will be briefly described prior to describing the disclosed example(s) in detail. The terms used herein have been selected as general terms which are widely used at present in consideration of the functions of the present disclosure, and this may be altered according to the intent of an operator skilled in the art, related practice, or introduction of new technology. In addition, in specific cases, certain terms may be arbitrarily selected by the applicant, and the meaning of the terms will be described in detail in a corresponding description of the example(s). Therefore, the terms used in the present disclosure should be defined based on the meaning of the terms and the overall content of the present disclosure rather than a simple name of each of the terms.

The singular forms “a,” “an,” and “the” as used herein are intended to include the plural forms as well, unless the context clearly indicates the singular forms. Further, the plural forms are intended to include the singular forms as well, unless the context clearly indicates the plural forms.

Further, throughout the description, when a portion is stated as “comprising (including)” a component, it is intended as meaning that the portion may additionally comprise (or include or have) another component, rather than excluding the same, unless specified to the contrary.

Further, the term “module” or “unit” used herein refers to a software or hardware component, and “module” or “unit” performs certain roles. However, the meaning of the “module” or “unit” is not limited to software or hardware. The “module” or “unit” may be configured to be in an addressable storage medium or configured to play one or more processors. Accordingly, as an example, the “module” or “unit” may include components such as software components, object-oriented software components, class components, and task components, and at least one of processes, functions, attributes, procedures, subroutines, program code segments, drivers, firmware, micro-codes, circuits, data, database, data structures, tables, arrays, or variables. Furthermore, functions provided in the components and the “modules” or “units” may be combined into a smaller number of components and “modules” or “units”, or further divided into additional components and “modules” or “units.”

According to an embodiment of the present disclosure, the “module” or “unit” may be implemented as a processor and a memory. The “processor” should be interpreted broadly to encompass a general-purpose processor, a Central Processing Unit (CPU), a microprocessor, a Digital Signal Processor (DSP), a controller, a microcontroller, a state machine, and so forth. Under some circumstances, the “processor” may refer to an application-specific integrated circuit (ASIC), a programmable logic device (PLD), a field-programmable gate array (FPGA), and so on. The “processor” may refer to a combination for processing devices, e.g., a combination of a DSP and a microprocessor, a combination of a plurality of microprocessors, a combination of one or more microprocessors in conjunction with a DSP core, or any other combination of such configurations. In addition, the “memory” should be interpreted broadly to encompass any electronic component that is capable of storing electronic information. The “memory” may refer to various types of processor-readable media such as random access memory (RAM), read-only memory (ROM), non-volatile random access memory (NVRAM), programmable read-only memory (PROM), erasable programmable read-only memory (EPROM), electrically erasable PROM (EEPROM), flash memory, magnetic or marking data storage, registers, etc. The memory is said to be in electronic communication with a processor if the processor can read information from and/or write information to the memory. The memory integrated with the processor is in electronic communication with the processor.

In the present disclosure, a “system” may refer to at least one of a server apparatus and a cloud apparatus, but is not limited thereto. For example, the system may include one or more server apparatus. In another example, the system may include one or more cloud apparatus. In still another example, the system may include both the server apparatus and the cloud apparatus operated in conjunction with each other.

In the present disclosure, “machine learning model” may include any model that is used for inferring an answer to a given input. According to an embodiment, the machine learning model may include an artificial neural network model including an input layer, a plurality of hidden layers, and an output layer. Each layer may include a plurality of nodes. The present disclosure describes a plurality of machine learning models such as a first machine learning model, a second machine learning model, a third machine learning model, etc. as separate machine learning models, but aspects are not limited thereto, and some or all of the plurality of machine learning models may be implemented as one machine learning model. In addition, in the present disclosure, the machine learning model may refer to an artificial neural network model, and the artificial neural network model may refer to the machine learning model.

In the present disclosure, a “display” may refer to any display apparatus associated with the computing apparatus. For example, it may refer to any display apparatus capable of displaying any information and data controlled by or provided from the computing apparatus.

In the present disclosure, “each of a plurality of A's” may refer to each of all components included in the plurality of A's, or may refer to each of some of the components included in the plurality of A's.

In the present disclosure, “implant” may refer to a procedure of replacing a missing tooth in dental treatment, and to a branch of medical field that involves the implantation of an artificial tooth to achieve a healthy mouth.

In the present disclosure, “nerve treatment” may refer to a procedure of removing the tissue when pulps are damaged due to gingivitis, periodontal diseases, etc., and filling a special material to restore a natural tooth so that the tooth functions without pain.

In the present disclosure, “dental image” may refer to any image, picture, etc. associated with diagnosis, dental treatment, examination, or procedure of periodontal diseases. For example, the “dental image” may include panoramic radiographic images, apicectomy images, computed tomography (CT) images, cone beam CT images, 3D CT images, intramural radiographic images, head standard radiographic images, etc., but is not limited thereto.

In the present disclosure, “nerve region of a tooth” may refer to any region for generating a guide for dental treatment. For example, the “nerve region of the tooth” may include a region (e.g., a square-shaped region) including a tooth and nerve tissue surrounding the tooth.

In the present disclosure, “guide data” may include data that may be an auxiliary indicator for the treatment of a tooth such as an implant or nerve treatment. For example, first guide data, which may be guide data for implant, may include a distance between a root tip and a bone level, a thickness and a length of a periodontal ligament, an amount of bones in the nerve region, etc., but is not limited thereto. As another example, second guide data, which may be guide data for nerve treatment, may include coordinates of a neural canal opening in the tooth, the number of neural canals, a length of the neural canal, a shape of the neural canal, etc., but is not limited thereto.

In the present disclosure, “patient information” may include information on a patient that may affect the nerve treatment. For example, this may include the patient's age, gender, nerve treatment history, presence of implant, and disease information, but are not limited thereto.

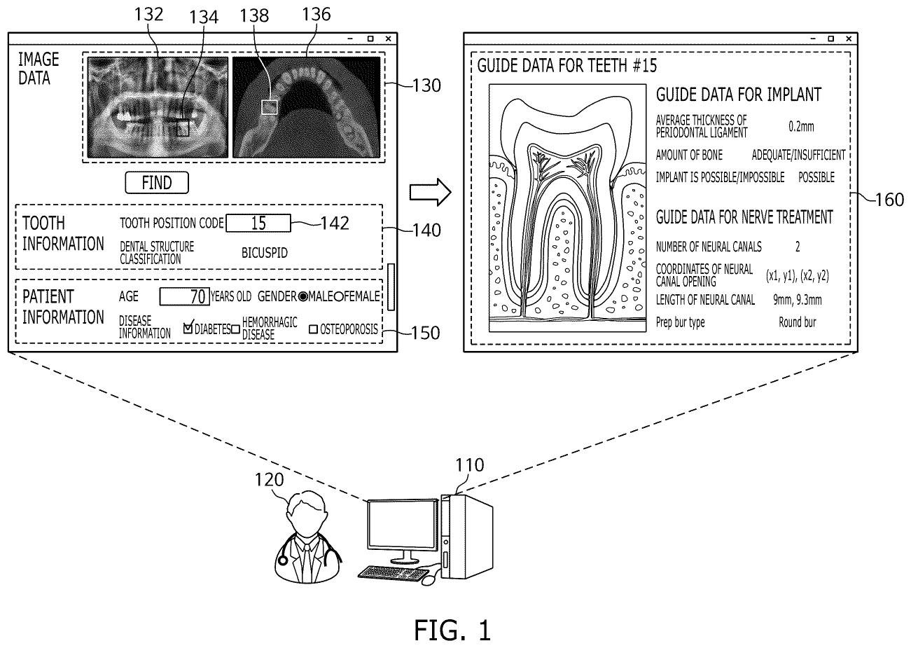

FIG. 1 is a diagram illustrating an example of providing guide data according to an embodiment of the present disclosure. As illustrated, a user 120 may check a guide for implant and/or nerve treatment through a display of a user terminal 110. In an embodiment, the user terminal 110 may receive a dental image from a photographing device, a storage device, etc. FIG. 1 illustrates a panoramic radiographic image as a dental image, but aspects are not limited thereto, and a dental image may include an apicectomy image, a computerized tomography (CT) image, a cone beam CT (CBCT) image, a 3D CT image, an intracranial radiation image, etc. Additionally or alternatively, the user terminal 110 may receive an image including a detected tooth region, or an image including an identified nerve region of a tooth.

The user terminal 110 may identify, among a plurality of nerve regions in a received dental image 130, a nerve region corresponding to received tooth information 140. The nerve region may refer to any region for generating a guide for dental treatment such as implant and/or nerve treatment. For example, the nerve region may be a region including a tooth and nerve tissue surrounding the tooth. With such a configuration, a region in the image that is unnecessary for guide generation may be removed, thereby improving processing speed and accuracy.

In an embodiment, the dental image 130 with an identified nerve region corresponding to a received position code 142 of the teeth may be displayed on the display of the user terminal 110. For example, as illustrated, nerve regions 134 and 138 may be displayed in the form of bounding boxes on a frontal image 132 and a tomographic image 136, respectively, and output on the display of the user terminal 110. According to an embodiment, the user 120 may modify the nerve regions. For example, the user 120 may move the position of the displayed bounding box in a drag-and-drop manner to modify an incorrectly identified nerve region. In another embodiment, the user 120 may select one or more nerve regions from among a plurality of nerve regions. In this case, the selected nerve region may be highlighted and displayed on the display of the user terminal 110.

Additionally, the user terminal 110 may receive patient information 150 associated with the dental image from the user 120. The patient information may include information on a patient that may affect the dental treatment. In the present disclosure, the patient's age, gender, nerve treatment history, implant status, and disease information are illustrated as examples of the patient information, but aspects are not limited thereto, and in some embodiments, the patient information may include surgical history, smoking status, bone quality information, family history information, etc.

The user terminal 110 may input the nerve region to the first machine learning model to generate segmentation data obtained by segmenting the tooth included in the nerve region and the nerve tissue surrounding the tooth, and input the segmentation data to the second machine learning model and/or the third machine learning model to generate guide data for implant and/or nerve treatment. At this time, the user terminal 110 may select, from among a plurality of first machine learning models, second machine learning models, and third machine learning models, a first machine learning model, a second machine learning model, and a third machine learning model, which may be associated with the received tooth position code, to generate the guide data. Additionally, if the patient information is received, the user terminal 110 may input the nerve region and the patient information to the first, second, and third machine learning models to generate guide data 160 for the tooth corresponding to the nerve region. The guide data 160 may include implant guide data and/or nerve treatment guide data. In an embodiment, if the user terminal 110 receives a request to output the first guide data indicating the implant guide from the user 120, the user terminal 110 may output the implant guide data for implant procedure. For example, the implant guide data may include data that can be an auxiliary indicator of the implant, such as average thickness and length of periodontal ligament, an amount of bone surrounding the tooth, a distance between an alveolar neural canal and a tooth, a distance between a root tip and a bone level, a length of a crown and a root, a state of a root canal, a state of the root, presence or absence of an apicectomy lesion, and a state of gingiva and alveolar bone around the periodontal ligament, and a treatment plan for implant may be established based on the implant guide data. In another embodiment, upon receiving a request to output the second guide data indicating a nerve treatment guide from the user 120, the user terminal 110 may output the nerve treatment guide data for the nerve treatment. For example, the nerve treatment guide data may include data that can serve as an auxiliary indicator for nerve treatment, such as coordinates of a neural canal opening in the tooth, the number of neural canals, a length of the neural canal and a shape of the neural canal, prep guide data, an angle of insertion of a prep bur, an amount of tooth removal, etc., and a treatment plan for nerve treatment may be established based on the nerve treatment guide data. In some embodiments, the guide data may be overlaid on each of the frontal image 132 and the tomographic image 136 displaying the nerve region of the tooth and output.

With such a configuration, the present disclosure may generate guide data for implant and/or nerve treatment based on the position code of the tooth and the dental image, thus providing a guide for objective and accurate dental treatment. In addition, in some embodiments of the present disclosure, the guide data may be generated in consideration of the patient information, thereby providing dental treatment guide data suitable for individual patients and improving the accuracy of implant and/or nerve treatment. By referring to the dental treatment guide data provided by the user terminal, the user 120 may establish the implant treatment plan and/or the nerve treatment plan or perform the implant procedure and/or the nerve treatment.

In the present disclosure, it has been described that the user terminal 110 receives and processes various information and provide the user 120 with the dental treatment guide data for implant and/or nerve treatment, but aspects are not limited thereto. For example, the user terminal 110 may transmit various received information and data to an external device (e.g., an information processing system), and the external device may generate the guide data based on the information and data received from the user terminal 110. In this case, the external apparatus may transmit the generated guide data to the user terminal 110. Additionally or alternatively, the user terminal 110 and the external device may be configured to process tasks for generating the guide data (e.g., operation task of the machine learning model, etc.) in a shared manner.

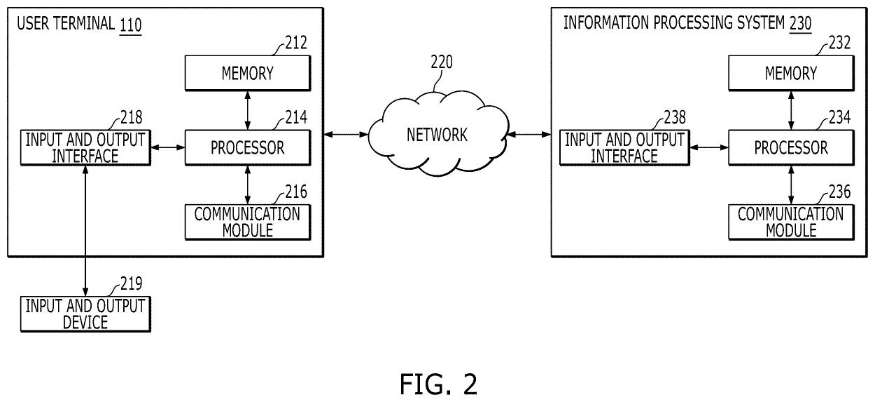

FIG. 2 is a block diagram illustrating an internal configuration of a user terminal 110 and an information processing system 230 according to an embodiment. The user terminal 110 may refer to any computing device that is capable of executing a guide application for dental treatment and performing wired/wireless communication, and may include, for example, a mobile phone terminal, a tablet terminal, a PC terminal, etc. As illustrated, the user terminal 110 may include a memory 212, a processor 214, a communication module 216, and an input and output interface 218. Likewise, the information processing system 230 may include a memory 232, a processor 234, a communication module 236, and an input and output interface 238. As illustrated in FIG. 2, the user terminal 110 and the information processing system 230 may be configured to communicate information and/or data through a network 220 using respective communication modules 216 and 236. In addition, an input and output device 219 may be configured to input information, data, etc. to the user terminal 110, or output information, data, etc. generated from the user terminal 110 through the input and output interface 218.

The memories 212 and 232 may include any non-transitory computer-readable recording medium. According to an embodiment, the memories 212 and 232 may include a permanent mass storage device such as random access memory (RAM), read only memory (ROM), disk drive, solid state drive (SSD), flash memory, etc. As another example, a non-destructive mass storage device such as ROM, SSD, flash memory, disk drive, etc. may be included in the user terminal 110 or the information processing system 230 as a separate permanent storage device that is distinct from the memory. In addition, the memory 212 and 232 may store an operating system and at least one program code (e.g., a code for the guide application for dental treatment).

These software components may be loaded from a computer-readable recording medium separate from the memories 212 and 232. Such a separate computer-readable recording medium may include a recording medium directly connectable to the user terminal 110 and the information processing system 230, and may include a computer-readable recording medium such as a floppy drive, a disk, a tape, a DVD/CD-ROM drive, a memory card, etc., for example. As another example, the software components may be loaded into the memories 212 and 232 through the communication modules 216 and 236 rather than the computer-readable recording medium. For example, at least one program may be loaded into the memories 212 and 232 based on a computer program (e.g., the guide application for dental treatment, etc.) installed by files provided by developers or a file distribution system for distributing an installation file of the application through the network 220.

The processors 214 and 234 may be configured to process the instructions of the computer program by performing basic arithmetic, logic, and input and output operations. The instructions may be provided to the processors 214 and 234 from the memories 212 and 232 or the communication modules 216 and 236. For example, the processors 214 and 234 may be configured to execute the received instructions according to a program code stored in a recording device such as the memories 212 and 232.

The communication modules 216 and 236 may provide a configuration or function for the user terminal 110 and the information processing system 230 to communicate with each other through the network 220, and may provide a configuration or function for the user terminal 110, the information processing system 230, etc. to communicate with another user terminal or another system (e.g., a separate cloud system, etc.). For example, a request or data (e.g., a request to generate guide related data, etc.) generated by the processor 214 of the user terminal 110 according to the program code stored in the recording device such as the memory 212 may be transmitted to the information processing system 230 through the network 220 under the control of the communication module 216. Conversely, a control signal or command provided under the control of the processor 234 of the information processing system 230 may be sent through the communication module 236 and the network 220 and received by the user terminal 110 through the communication module 216 of the user terminal 110. For example, the user terminal 110 may receive, from the information processing system 230, an image with an identified nerve region corresponding to the tooth position code, guide data for dental treatment, etc. through the communication module 216.

The input and output interface 218 may be a means for interfacing with the input and output device 219. As an example, the input device may include a device such as a camera including an audio sensor and/or an image sensor, a keyboard, a microphone, a mouse, etc., and the output device may include a device such as a display, a speaker, a haptic feedback device, etc. As another example, the input and output interface 218 may be a means for interfacing with a device such as a touch screen, etc. that incorporates a configuration or function for performing inputting and outputting. While FIG. 2 illustrates that the input and output device 219 is not included in the user terminal 110, aspects are not limited thereto, and the input and output device 219 may be configured as one device with the user terminal 110. In addition, the input and output interface 238 of the information processing system 230 may be a means for interfacing with a device (not illustrated) for inputting or outputting, which may be connected to or included in the information processing system 230. While FIG. 2 illustrates the input and output interfaces 218 and 238 as the components configured separately from the processors 214 and 234, aspects are not limited thereto, and the input and output interfaces 218 and 238 may be configured to be included in the processors 214 and 234.

The user terminal 110 and the information processing system 230 may include more components than those illustrated in FIG. 2. Meanwhile, most of the related components may not necessarily require exact illustration. In an embodiment, the user terminal 110 may be implemented to include at least a part of the input and output device 219 described above. In addition, the user terminal 110 may further include another component such as a transceiver, a global positioning system (GPS) module, a camera, various sensors, a database, etc. For example, if the user terminal 110 is a smartphone, it may include components generally included in the smartphone, and for example, it may be implemented such that various components such as an acceleration sensor, a gyro sensor, a microphone module, a camera module, various physical buttons, buttons using a touch panel, input and output ports, a vibrator for vibration, etc. are further included in the user terminal 110.

According to an embodiment, the processor 214 of the user terminal 110 may be configured to operate a guide application for dental treatment. In this case, a program code associated with that application may be loaded into the memory 212 of the user terminal 110. While the application is running, the processor 214 of the user terminal 110 may receive information and/or data provided from the input and output device 219 through the input and output interface 218 or receive information and/or data from the information processing system 230 through the communication module 216, and process the received information and/or data and store it in the memory 212. In addition, such information and/or data may be provided to the information processing system 230 through the communication module 216.

While a guide application for dental treatment, etc. is running, the processor 214 may receive voice data, text, images, videos, etc. input or selected through an input device such as a touch screen, a keyboard, a camera including an audio sensor and/or an image sensor, a radiographic device, a tomography device, a microphone, etc., connected to the input and output interface 218, and store the received voice data, text, images, and/or videos, etc. in the memory 212 or provide the same to the information processing system 230 through the communication module 216 and the network 220. In an embodiment, the processor 214 may receive a dental image, a request to generate guide data for dental treatment, etc. through the input device 219 such as a keyboard or a mouse, and provide the same to the information processing system 230 through the communication module 216 and the network 220.

The processor 214 of the user terminal 110 may transmit and output the information and/or data to the input and output device 219 through the input and output interface 218. For example, the processor 214 of the user terminal 110 may output the processed information and/or data through the output device 219 such as a device (e.g., a touch screen, a display, etc.) capable of outputting a display, a device (e.g., speaker) capable of outputting a voice, etc. In an embodiment, the processor 214 may display the guide data for dental treatment on the display of the user terminal 110. Additionally, the processor 214 may convert text information of the guide data for dental treatment into voice and output the voice through the speaker of the user terminal 110.

The processor 234 of the information processing system 230 may be configured to manage, process, and/or store information, data, etc. received from a plurality of user terminals 110, a plurality of external systems, etc. The information and/or data processed by the processor 234 may be provided to the user terminals 110 through the communication module 236 and the network 220. In an embodiment, the processor 234 of the information processing system 230 may identify a nerve region from the received dental image and generate the guide data for dental treatment based on the position code of the tooth and the patient information. In addition, the generated guide data for dental treatment may be provided to the user terminal 110 through the communication module 236 and the network 220.

For example, the processor 234 may input the dental image to the first machine learning model, of the plurality of first machine learning models, associated with the received tooth position code to generate the segmentation data, and input the generated segmentation data to the second machine learning model, of the plurality of second machine learning models, associated with the received tooth position code to generate the guide data for the implant.

In another example, the processor 234 may input the dental image to the first machine learning model, of the plurality of first machine learning models, associated with the received tooth position code to generate the segmentation data, and input the generated segmentation data to the third machine learning model, of the plurality of third machine learning models, associated with the received tooth position code to generate the guide data for dental treatment such as nerve treatment.

The internal configurations of the user terminal and the information processing system illustrated in FIG. 2 are merely examples, and aspects are not limited thereto. In some embodiments, some of the illustrated configurations may be omitted. For example, an embodiment including only the information processing system 230 without the user terminal 110 is possible, and in this case, the operations performed by the processor 214 of the user terminal may be performed by the processor 234 of the information processing system. In addition, an embodiment including only the user terminal 110 without the information processing system 230 is also possible, and in this case, the operations performed by the processor 234 of the information processing system may be performed by the processor 214 of the user terminal.

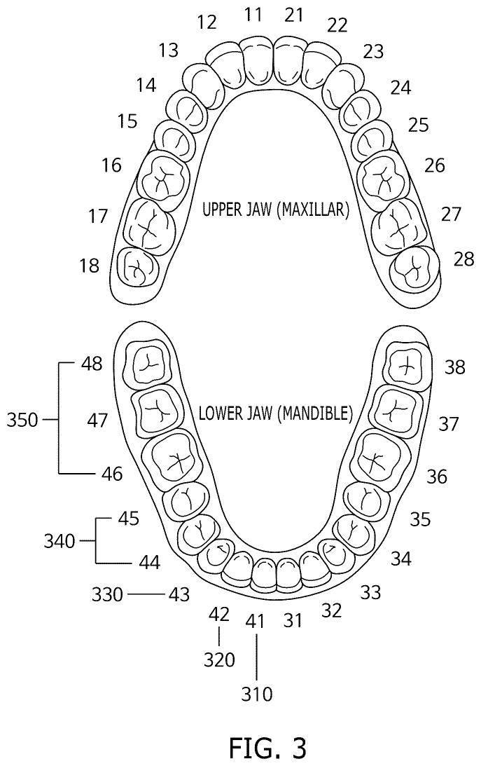

FIG. 3 is a diagram illustrating a position code of each of a plurality of teeth. The position code of the tooth is a number used by dentists to identify the position of the tooth, and may be assigned to each of the plurality of teeth such that the numbers do not overlap each other. The entire teeth can be classified into maxilla, mandible, right teeth, and left teeth, each being divided into central incisor, lateral incisor, canine, bicuspid, and molar structures.

As illustrated, the position codes of central incisor structures 310 may be 11, 21, 31, and 41, the position codes of lateral incisor structures 320 may be 12, 22, 32, and 42, the position codes of canine structures 330 may be 13, 23, 33, and 43, the position codes of bicuspid structures 340 may be 14, 15, 24, 25, 34, 35, 44, and 45, and the position codes of molar structures 350 may be 16, 17, 18, 26, 27, 28, 36, 37, 38, 46, 47, and 48. In the present disclosure, the position codes of the teeth may be used for selecting a tooth for nerve treatment or for selecting the first machine learning model, the second machine learning model, and/or the third machine learning model associated with the tooth structure corresponding to the position code.

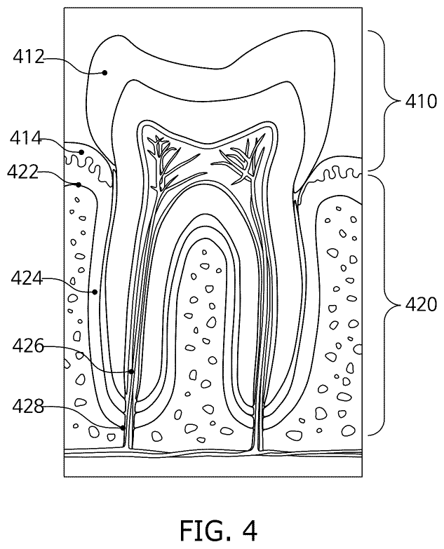

FIG. 4 is a diagram illustrating a nerve region of a tooth according to an embodiment of the present disclosure; The nerve region including a tooth may be divided into a crown 410 and a root 420, in which the crown 410 may include an enamel 412 and a gingiva 414, and the root 420 may include a bone level 422, a periodontal ligament 424, a neural canal (root canal) 426, and a root tip 428. The enamel 412 is a white-colored tissue formed of inorganic substances and minerals and positioned on an uppermost part of the crown 410 of the teeth, and it may refer to tissue that is prepared for nerve treatment along with dentin. The gingiva 414 is a soft tissue surrounding the tooth and may refer to a tissue protecting the tooth.

The bone level 422 may refer to the upper tissue of the alveolar bone, which is a gum bone supporting the tooth, and the implant guide data may be generated using the length from the bone level to the root tip, that is, to the apex of the tooth's root. The periodontal ligament 424 is a tissue that attaches teeth and gums with a strong bonding force, and may refer to a tissue that buffers the pressure occurred when chewing food, and the implant guide data may be generated based on the average thickness and length of the periodontal ligament 424. The neural canal (root canal) 426 is an anatomical space present in the root 420, and includes pulps which are tissues constituting the dentin part of the tooth. The nerve treatment guide data may be generated based on the shape, length, number of the neural canals, coordinates of the neural canal opening, etc. In the present disclosure, the forms of tissues constituting the nerve region of a tooth may be used for generating the implant guide data and/or the nerve treatment guide data for dental treatment.

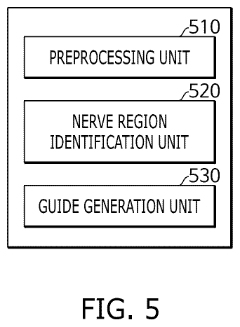

FIG. 5 is a block diagram illustrating a detailed configuration of a processor according to an embodiment of the present disclosure. As illustrated, the processor (e.g., the processor 234 of the information processing system 230) may include a preprocessing unit 510, a nerve region identification unit 520, and a guide generation unit 530. The processor may communicate with an external device (e.g., user terminal, external device, etc.) and receive information necessary for generating a guide. For example, the guide may be first guide data for implant treatment. As another example, the guide may be second guide data for nerve treatment.

The preprocessing unit 510 may perform preprocessing on the dental image. In an embodiment, the preprocessing unit 510 may crop a part of the image. Additionally or alternatively, the preprocessing unit 510 may resample such that the length of one pixel in the image corresponds to a predetermined length. In addition, the preprocessing unit 510 may perform any preprocessing on the received dental image in a form suitable for identifying a nerve region or generating guide data. In some embodiments, the preprocessing unit 510 may be omitted.

the nerve region identification unit 520 may identify a plurality of nerve regions in the received dental image, and then identify a nerve region corresponding to the received position code of the tooth. The nerve region of the tooth may include a tooth and a nerve tissue around the tooth. According to some embodiments, the nerve region identification unit 520 may identify a plurality of nerve regions from the received dental image using a predetermined algorithm and/or machine learning model (e.g., a convolutional neural network (CNN) among artificial neural network models). Additionally or alternatively, the nerve region identification unit 520 may detect a plurality of tooth regions from the received dental image and then identify a nerve region of the tooth for implant procedure and/or nerve treatment based on the detected plurality of tooth regions and the received tooth position code. According to some embodiments, the nerve region identification unit 520 may detect a plurality of tooth regions from the received dental image using a predetermined algorithm and/or machine learning model, and identify a nerve region of a tooth for implant procedure and/or nerve treatment based on the received position code of the tooth. By performing one more operation of detecting the tooth region from the dental image, the accuracy of identifying the nerve region can be improved, and a more accurate guide can be provided. In this case, the nerve region identification unit 520 may use a machine learning model to detect the tooth region and use a separate machine learning model to identify the nerve region based on the detected tooth region, or may use a single machine learning model.

The nerve region identification unit 520 may generate segmentation data based on the position code of the tooth for dental treatment such as implant and/or nerve treatment, and a nerve region corresponding to the position code. According to an embodiment, the nerve region identification unit 520 may input the corresponding nerve region of the tooth to the first machine learning model, of the plurality of first machine learning models (e.g., CNN, etc. of artificial neural network models), associated with the received tooth position code to generate segmentation data.

For example, if the position code of the tooth for treatment is “48”, a first machine learning model associated with the right molar of the mandible may be selected. The segmentation data may refer to data obtained by dividing shapes of a tooth and the tissues constituting the nerve region of the tooth, and each tissue may be expressed differently through expressions such as brightness, color, texture, etc. For example, the segmentation data may refer to data obtained by dividing a nerve region including the enamel, gingiva, periodontal ligament, neural canal, root tip, neural canal opening, etc. according to an axial plane, coronal plane, and sagittal plane, and even for the same nerve tissue, the data may be generated with different expressions such as brightness, color, and texture according to thickness, length, size, etc.

The guide generation unit 530 may output the implant guide data and/or the nerve treatment guide data according to a request to output guide data received from the user terminal. According to an embodiment, upon receiving a request to output guide data for implant treatment from the user terminal, the guide generation unit 530 may input segmentation data of a corresponding tooth to a second machine learning model, of the plurality of second machine learning models, associated with a received tooth position code to output first guide data indicating implant guide data. For example, the first guide data may include data that can be an auxiliary indicator of the implant, such as an average thickness and length of the periodontal ligament included in the nerve region, an amount of bone surrounding the tooth, a distance between the alveolar neural canal and the tooth, a distance between the root tip and the bone level, a length of the crown and the root, a state of the root canal, a state of the root, presence or absence of the apicectomy lesion, and a state of the gingiva and alveolar bone around the periodontal ligament.

According to another embodiment, upon receiving a request to output guide data for nerve treatment from the user terminal, the guide generation unit 530 may input segmentation data of a corresponding tooth to a third machine learning model, of the plurality of third machine learning models, associated with a received tooth position code to output second guide data indicating nerve treatment guide data. For example, the second guide data may include data that can serve as auxiliary indicators for nerve treatment, such as coordinates of the neural canal opening in the tooth, the number of neural canals, a length of the neural canal and a shape of the neural canal, prep guide data, an angle of insertion of the prep bur, and an amount of tooth removal. In this case, the guide data may be overlaid on the dental image and output.

According to an embodiment, the preprocessing unit 510, the nerve region identification unit 520, and the guide generation unit 530 may be included in the processor of the information processing system. In some other embodiments, some or all of the preprocessing unit 510, the nerve region identification unit 520, or the guide generation unit 530 may be included in the processor of the user terminal.

Although the configurations of the processor have been classified by functions and described in FIG. 5, it does not necessarily mean that they are physically divided. For example, it is described above that the preprocessing unit 510, the nerve region identification unit 520, and the guide generation unit 530 are divided, but this is to help understand the disclosure and aspects are not limited thereto. For example, the nerve region identification unit 520 and the guide generation unit 530 may be configured to include one artificial neural network model or a plurality of different artificial neural network models.

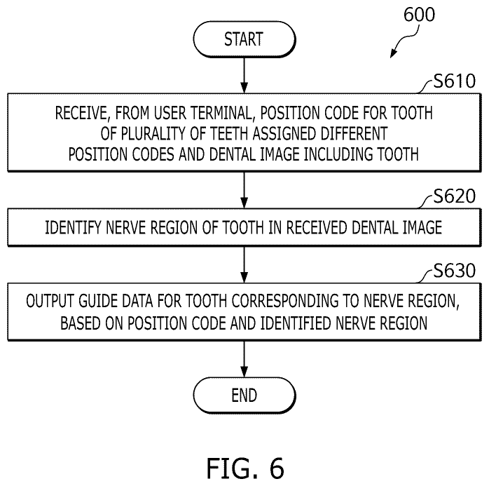

FIG. 6 is a flowchart illustrating a method for generating a guide for dental treatment according to an embodiment of the present disclosure. According to an embodiment, a method 600 for generating a guide for dental treatment may be performed by a processor (e.g., processor of the user terminal and/or at least one processor of the information processing system, etc.). As illustrated, the method 600 for generating the guide for dental treatment may be initiated by the processor receiving, from the user terminal, a position code for a tooth of a plurality of teeth assigned different position codes, and a dental image including the tooth, at S610. For example, the dental image may be received from the user terminal 110, an external device, etc., or may be received from an internal memory.

The processor may identify a nerve region of the tooth in the received dental image, at S620. According to an embodiment, the processor may identify a plurality of tooth regions from the received dental image using a predetermined algorithm and/or a machine learning model (e.g., CNN of the artificial neural network models, etc.). For example, the processor may detect a plurality of tooth regions from the dental image and identify, based on the received tooth position code, a nerve region from among the plurality of tooth regions. In this case, the nerve region of the tooth includes the tooth and the nerve tissue around the tooth, and one or more machine learning models may be used for detection of the nerve region. Additionally, the processor may select, from among the plurality of first machine learning models, the first machine learning model associated with the position code of the tooth, and input the nerve region of the tooth into the selected first machine learning model to perform segmentation on the nerve region. For example, the processor may perform segmentation so as to divide the shape of the tooth and tissues constituting the nerve region of the tooth and express the result differently.

The method 600 may be terminated by the processor outputting guide data for the tooth corresponding to the nerve region, based on the position code and the identified nerve region, at S630. According to an embodiment, the processor may receive, from the user terminal, a request to output the first guide data, that is, the guide data for implant treatment. Upon receiving the request to output the first guide data, the processor may select a second machine learning model associated with the position code of the tooth from among the plurality of second machine learning models, and input the segmentation performed on the nerve region to the selected second machine learning model to output the first guide data. For example, the first guide data may include data that can be an auxiliary indicator of the implant, such as an average thickness and length of the periodontal ligament included in the nerve region, an amount of bone surrounding the tooth, a distance between the alveolar neural canal and the tooth, a distance between the root tip and the bone level, a length of the crown and the root, a state of the root canal, a state of the root, presence or absence of the apicectomy lesion, and a state of the gingiva and alveolar bone around the periodontal ligament, but is not limited thereto.

According to another embodiment, the processor may receive, from the user terminal, a request to output second guide data, which is the guide data for nerve treatment. In response to receiving the request to output the second guide data, the processor may select the third machine learning model associated with the position code of the tooth from among the plurality of third machine learning models, and input the segmentation performed on the nerve region to the selected third machine learning model to output the second guide data. For example, the second guide data may include data that can serve as auxiliary indicators for nerve treatment, such as coordinates of the neural canal opening in the tooth, the number of neural canals, a length of the neural canal and a shape of the neural canal, prep guide data, an angle of insertion of the prep bur, and an amount of tooth removal, but is not limited thereto.

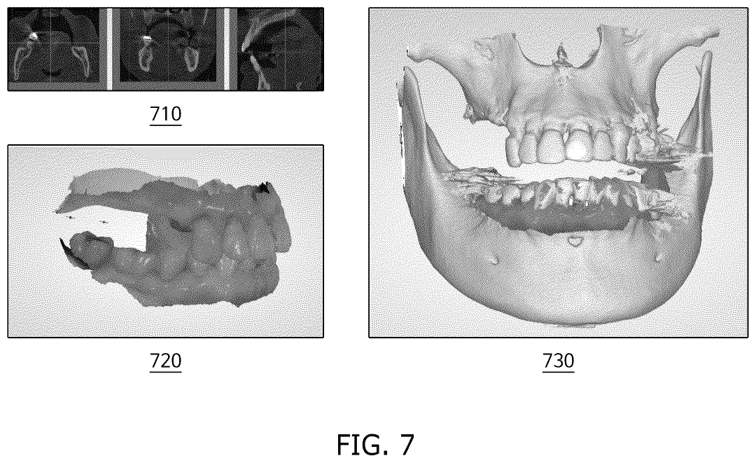

FIG. 7 illustrates an example of a dental image according to an embodiment of the present disclosure. According to an embodiment, the processor may receive dental images 710, 720, and 730. For example, the dental images 710, 720, and 730 may be a CT image 710, an oral scan image 720, and a matching image 730, but are not limited thereto, and panoramic images, apicectomy images, computed tomography (CT) images, cone beam CT (CBCT) images, 3D CT images, and intravenous radiographic images may be used as the dental images. The processor may identify a nerve region corresponding to the position code of the teeth based on the dental images 710, 720, and 730, and overlay the guide data on the dental images 710, 720, and 730 and output the same.

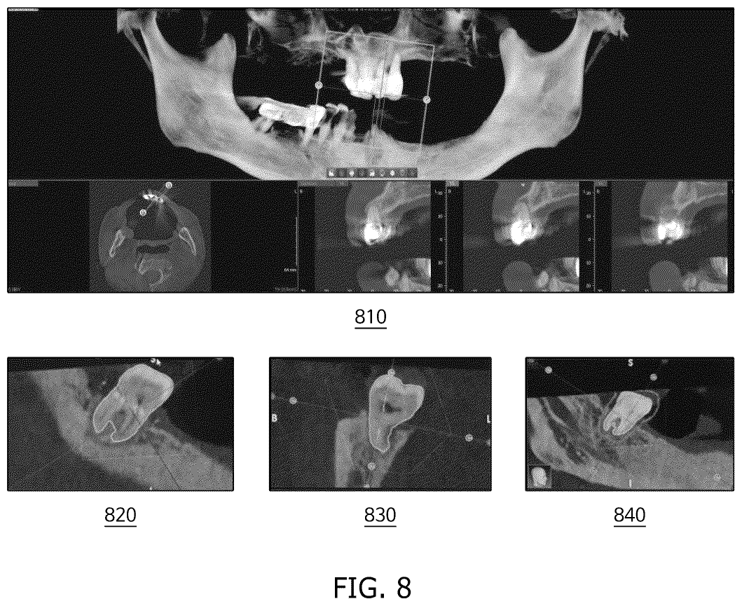

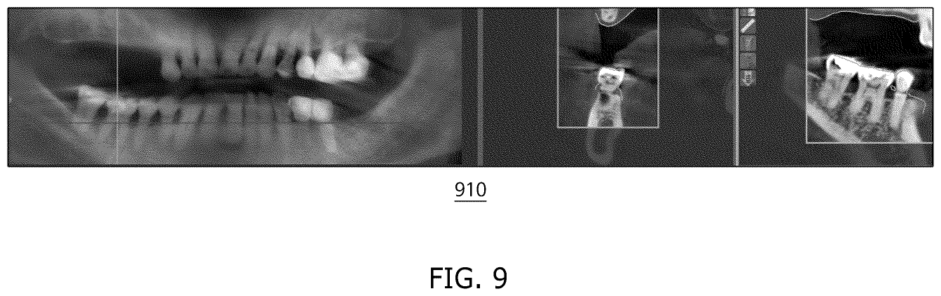

FIGS. 8 and 9 are diagrams illustrating an example in which segmentation is performed according to an embodiment. According to an embodiment, the processor may perform segmentation on the identified nerve region in dental images 810 and 910. The segmentation may refer to dividing teeth and tissues included in the nerve region, and may be performed from the perspective of a horizontal plane 820, a sagittal plane 830, and a coronal plane 840. For example, the segmentation may refer to expressing tissues that make up the nerve region, such as the enamel, gingiva, periodontal ligament, neural canal, root tip, neural canal opening, etc. included in the nerve region differently in terms of brightness, color, texture, etc. Additionally, the processor may compare the size, length, and shape of a tissue included in the nerve region with a predetermined threshold, and if it is determined that there is a difference of more than a certain error, may segment only the corresponding tissue and separately output the result to the user terminal.

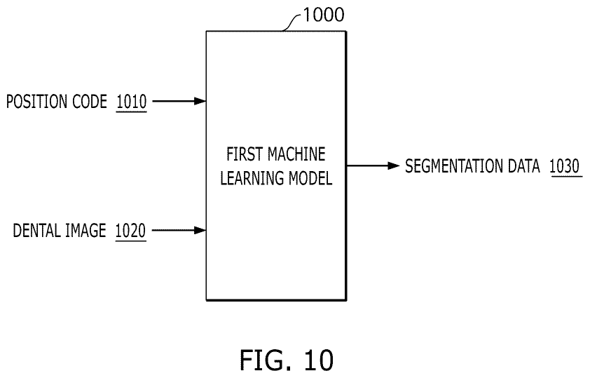

FIG. 10 is a diagram illustrating an example of inputting a position code and a dental image to a machine learning model to generate segmentation data according to an embodiment of the present disclosure. As described above, the processor may receive a position code 1010. The position code 1010 is a code assigned to each of the plurality of teeth such that the codes do not overlap each other, and in response to receiving the position code of a tooth for dental treatment such as implant and/or nerve treatment from the user, a first machine learning model 1000 for generating segmentation data 1030 may be selected. For example, the first machine learning model may be selected according to at least one tooth structure of the central incisor, lateral incisor, canine, bicuspid, and molar corresponding to the received position code. In this case, the first machine learning model may be trained to output the segmentation data 1030 according to the structure of the tooth corresponding to the position code.

According to an embodiment, the processor may use the first machine learning model 1000 to generate the segmentation data 1030 based on the position code 1010 and a dental image 1020. For example, the first machine learning model 1000 may receive the position code 1010 and the dental image 1020 and output the segmentation data 1030. In an embodiment, the segmentation data 1030 may include data obtained by dividing a tooth and the shape of tissues constituting a nerve region of the tooth. For example, the segmentation data may include data obtained by dividing a nerve region including the enamel, gingiva, periodontal ligament, neural canal, root tip, neural canal opening, etc. according to axial plane, coronal plane, and sagittal plane, but is not limited thereto.

The first machine learning model 1000 may be trained with various training data. For example, the first machine learning model 1000 may be trained with training data including a position code, dental image and segmentation data pair. The segmentation data included in the training data may include data that differently expresses tissues in the nerve region such as enamel, gingiva, periodontal ligament, neural canal, root tip, neural canal opening, etc. In an embodiment, the processor may receive segmentation data modified by the user. In this case, the first machine learning model 1000 may be re-trained based on the modified segmentation data.

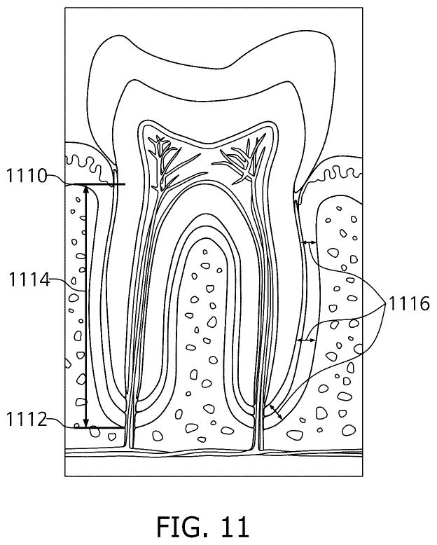

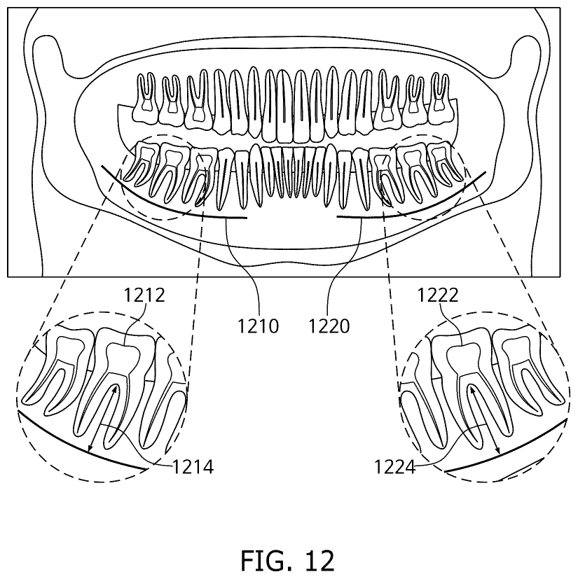

FIGS. 11 and 12 are diagrams illustrating an example of implant guide data according to an embodiment of the present disclosure. According to an embodiment, the processor may output the first guide data, that is, the guide data for implant treatment, based on the segmentation data. The first guide data may refer to data that may be an auxiliary indicator for implant procedure, and it is possible to determine whether the implant can be performed/is possible, when to perform the implant, etc. based on the first guide data. For example, the first guide data may include a distance 1114 between a root tip 1112 and a bone level 1110 in the nerve region, an average thickness 1116 and length of the periodontal ligament, an amount of bone surrounding the tooth, a distance between alveolar neural canals 1210 and 1220 and teeth 1212 and 1222, a length of the crown and the root, a state of the root canal, a state of the root, presence or absence of the apicectomy lesion, a state of the gingiva and alveolar bone around the periodontal ligament, etc.

In an embodiment, the processor may measure the distance 1114 between the root tip 1112 and the bone level 1110 and an average of thicknesses 1116 of the periodontal ligament and a distance thereof, and if the measured values are out of a predetermined range value, output the resultant value such as whether the implant can be performed/is possible on the user terminal together with the first guide data.

In another embodiment, the processor may measure distances 1214 and 1224 between the alveolar neural canals 1210 and 1220 and the teeth 1212 and 1222, and if the measured distances 1214 and 1224 are out of a predetermined range value, output the resultant value such as when to perform the implant, etc. on the user terminal together with the first guide data. For example, since the distance 1214 between the the right molar 1212 of the mandible with a tooth position code “47” and the alveolar neural canal 1210 is shorter than the distance 1224 between the left molar 1222 of the mandible with a tooth position code “37” and the alveolar neural canal 1220, the time to perform the implant of the right molar 1212 of the mandible may be earlier than that of the left molar 1222 of the mandible. The distance between the tooth and the alveolar neural canal may be redefined as the amount of bone surrounding the tooth and output as the first guide data.

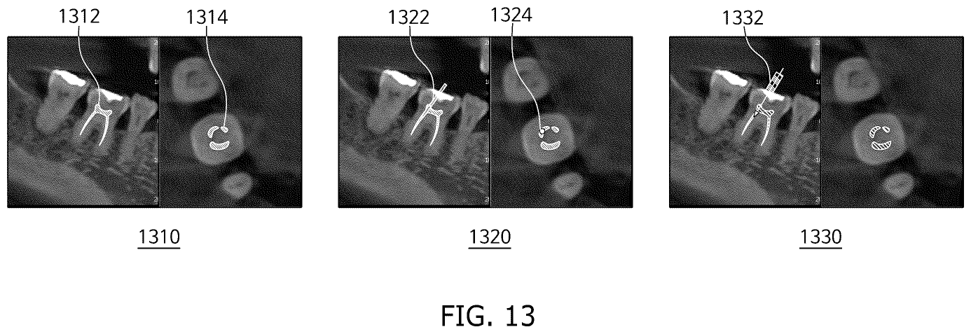

FIG. 13 is a diagram illustrating an example of nerve treatment guide data according to an embodiment of the present disclosure. According to an embodiment, the processor may output the second guide data, that is, the guide data for nerve treatment, based on the segmentation data. The second guide data may refer to data that may be an auxiliary indicator for nerve treatment, and it is possible to establish the treatment guide for nerve treatment based on the second guide data. For example, the second guide data may include coordinates of the neural canal opening in the tooth, the number of neural canals, a length of the neural canal and a shape of the neural canal, an amount of tooth removal, etc.

In another embodiment, the processor may output prep guide data as the second guide data. The prep may refer to removing areas of a tooth to reach the neural canal opening when performing nerve treatment, and the prep guide data may include a prep bur selection criterion, a prep bur insertion angle, a prep bur mm, a prep bur shape, etc., but aspects are not limited thereto. The processor may generate an image 1310 of segmented neural canals 1312 and 1314 from the received dental image. An image 1320 identifying an angle of an endodontic file 1322 to be inserted into the neural canal and a neural canal opening 1324 may be generated based on the shape of the neural canal. Based on the generated image 1320, the position and angle at which a prep bur 1332 is inserted to remove areas of the tooth may be overlaid (1330) together with the prep guide data and output to the user terminal.

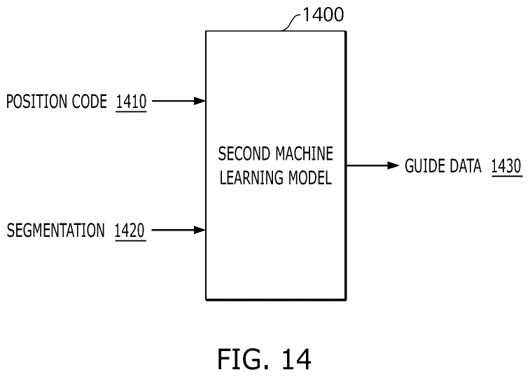

FIG. 14 is a diagram illustrating an example of inputting a position code and segmentation to generate first guide data according to an embodiment of the present disclosure. As described above, the processor may receive a position code 1410. In response to receiving the position code 1410 from the user, a second machine learning model 1400 for generating guide data 1430 for the implant may be selected. For example, the second machine learning model may be selected according to at least one tooth structure of the central incisor, lateral incisor, canine, bicuspid, and molar corresponding to the received position code. In this case, the first machine learning model may be trained to output the guide data 1430 according to the structure of the tooth corresponding to the position code.

According to an embodiment of the present disclosure, the processor may use the second machine learning model 1400 to generate the guide data 1430 based on the position code 1410 and segmentation data 1420. For example, the second machine learning model 1400 may receive the position code 1410 and the segmentation data 1420 and output the guide data 1430. In an embodiment, the guide data 1430 may include first guide data, which is guide data for implant treatment. For example, the first guide data may include a distance 1114 between a root tip 1112 and a bone level 1110 in the nerve region, an average thickness 1116 and length of the periodontal ligament, an amount of bone surrounding the tooth, a distance between alveolar neural canals 1210 and 1220 and teeth 1212 and 1222, a length of the crown and the root, a state of the root canal, a state of the root, presence or absence of the apicectomy lesion, a state of the gingiva and alveolar bone around the periodontal ligament, etc.

The second machine learning model 1400 may be trained with various training data. For example, the second machine learning model 1400 may be trained with training data including the position code 1410, the segmentation data 1420 and the guide data 1430 pairs. The guide data 1430 included in the training data may include the first guide data and the second guide data. In an embodiment, the processor may receive guide data modified by the user. In this case, the second machine learning model 1400 may be re-trained based on the modified guide data 1430.

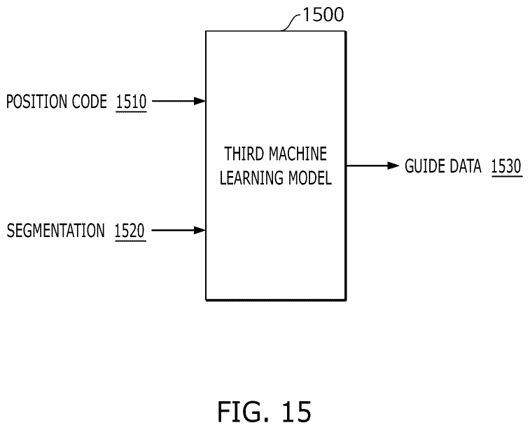

FIG. 15 is a diagram illustrating an example of inputting a position code and segmentation to generate second guide data according to an embodiment of the present disclosure. As described above, the processor may receive a position code 1510. In response to receiving the position code 1510 from the user, a third machine learning model 1500 for generating guide data 1530 for dental treatment such as nerve treatment may be selected. For example, the third machine learning model may be selected according to at least one tooth structure of the central incisor, lateral incisor, canine, bicuspid, and molar corresponding to the received position code. In this case, the third machine learning model may be trained to output the guide data 1530 according to the structure of the tooth corresponding to the position code.

According to an embodiment of the present disclosure, the processor may use the third machine learning model 1500 to generate the guide data 1530 based on the position code 1510 and segmentation data 1520. For example, the third machine learning model 1500 may receive the position code 1510 and the segmentation data 1520 and output the guide data 1530. In an embodiment, the guide data 1530 may include second guide data, which is guide data for nerve treatment. For example, the second guide data may include prep guide data, coordinates of a neural canal opening in the tooth, the number of neural canals, a length of the neural canal, a shape of the neural canal, etc.

The third machine learning model 1500 may be trained with various training data. For example, the third machine learning model 1500 may be trained with training data including the position code 1510, the segmentation data 1520 and the guide data 1530 pairs. The guide data 1530 included in the training data may include the second guide data. In an embodiment, the processor may receive guide data modified by the user. In this case, the third machine learning model 1500 may be re-trained based on the modified guide data 1530.

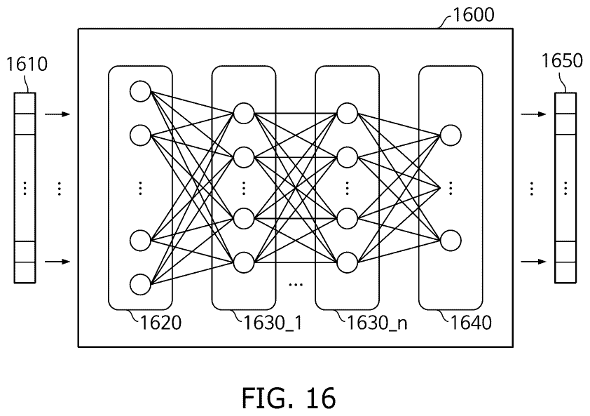

FIG. 16 is a diagram illustrating an example of an artificial neural network model according to an embodiment of the present disclosure. In machine learning technology and cognitive science, an artificial neural network model 1600 as an example of the machine learning model refers to a statistical learning algorithm implemented based on a structure of a biological neural network, or to a structure that executes such algorithm.

According to an embodiment, the artificial neural network model 1600 may represent a machine learning model that acquires a problem solving ability by repeatedly adjusting the weights of synapses by the nodes that are artificial neurons forming the network through synaptic combinations as in the biological neural networks, thus training to reduce errors between a target output corresponding to a specific input and a deduced output. For example, the artificial neural network model 1600 may include any probability model, neural network model, etc., that is used in artificial intelligence training methods such as machine learning and deep learning.

According to an embodiment, the nerve region identification unit and the guide generation unit may be generated in the form of the machine learning model, that is, in the form of the artificial neural network model 1600. For example, a plurality of artificial neural network models 1600 may be configured to generate segmentation data and/or guide data for each structure of the teeth structure including central incisor, lateral incisor, canine, bicuspid, and molar. The artificial neural network model 1600 corresponding to the received tooth position code may be selected from among the plurality of artificial neural network models 1600, and a nerve region may be input to generate the segmentation data and/or the guide data. In another example, the artificial neural network model 1600 may be configured to receive the position code, the dental image, and the patient information and generate the segmentation data and/or the guide data considering the patient information.

The artificial neural network model 1600 is implemented as a multilayer perceptron (MLP) formed of multilayer nodes and connections therebetween. The artificial neural network model 1600 may be implemented using one of various artificial neural network model structures including the MLP. As illustrated in FIG. 16, the artificial neural network model 1600 includes an input layer 1620 to receive an input signal or data 1660 from the outside, an output layer 1640 to output an output signal or data 1650 corresponding to the input data, and (n) number of hidden layers 1630_1 to 1630_n (where n is a positive integer) positioned between the input layer 1620 and the output layer 1640 to receive a signal from the input layer 1620, extract the features, and transmit the features to the output layer 1640. In an example, the output layer 1640 receives signals from the hidden layers 1630_1 to 1630_n and outputs the signals to the outside.

The method for training the artificial neural network model 1600 includes the supervised learning that trains to optimize for solving a problem with inputs of teacher signals (correct answers), and the unsupervised learning that does not require a teacher signal. The information processing system may train, by the supervised and/or unsupervised learning, the artificial neural network model 1600 that is trained to generate the segmentation data and/or the guide data from the position codes and the dental image. The trained artificial neural network model 1600 may be stored in a memory of the information processing system.

According to an embodiment, the input variables of the artificial neural network model 1600 may be the position code of the tooth and the dental image. Additionally, the input variable of the artificial neural network model 1600 may include the patient information, etc. That is, a vector indicating or characterizing the position code, the image, the patient information, etc. described above may be input through the input layer 1620. As described above, if the input variable described above is input through the input layer 1620, the output variable output from the output layer 1640 of the artificial neural network model 1600 may be a vector indicating or characterizing the segmentation data. According to another embodiment, the input variable of the artificial neural network model 1600 may be the position code and the segmentation data of the tooth. As described above, if the input variable described above is input through the input layer 1620, the output variable output from the output layer 1640 of the artificial neural network model 1600 may be a vector indicating or characterizing the guide data.

As described above, the input layer 1620 and the output layer 1640 of the artificial neural network model 1600 are respectively matched with a plurality of output variables corresponding to a plurality of input variables, and as the synaptic values between nodes included in the input layer 1620, and the hidden layers 1630_1 to 1630_n, and the output layer 1640 are adjusted, training can be processed to extract a correct output corresponding to a specific input. Through this training process, the features hidden in the input variables of the artificial neural network model 1600 may be confirmed, and the synaptic values (or weights) between the nodes of the artificial neural network model 1600 may be adjusted so as to reduce the errors between the output variable calculated based on the input variable and the target output. The segmentation data and/or the guide data may be output using the artificial neural network model 1600 trained as described above.

The method described above may be provided as a computer program stored in a computer-readable recording medium for execution on a computer. The medium may be a type of medium that continuously stores a program executable by a computer, or temporarily stores the program for execution or download. In addition, the medium may be a variety of writing means or storage means having a single piece of hardware or a combination of several pieces of hardware, and is not limited to a medium that is directly connected to any computer system, and accordingly, may be present on a network in a distributed manner. An example of the medium includes a medium configured to store program instructions, including a magnetic medium such as a hard disk, a floppy disk, and a magnetic tape, an optical medium such as a CD-ROM and a DVD, a magnetic-optical medium such as a floptical disk, and a ROM, a RAM, a flash memory, etc. In addition, other examples of the medium may include an app store that distributes applications, a site that supplies or distributes various software, and a recording medium or a storage medium managed by a server.

The methods, operations, or techniques of the present disclosure may be implemented by various means. For example, these techniques may be implemented in hardware, firmware, software, or a combination thereof. Those skilled in the art will further appreciate that various illustrative logical blocks, modules, circuits, and algorithm steps described in connection with the disclosure herein may be implemented in electronic hardware, computer software, or combinations of both. To clearly illustrate this interchangeability of hardware and software, various illustrative components, blocks, modules, circuits, and steps have been described above generally in terms of their functionality. Whether such a function is implemented as hardware or software varies according to design requirements imposed on the particular application and the overall system. Those skilled in the art may implement the described functions in varying ways for each particular application, but such implementation should not be interpreted as causing a departure from the scope of the present disclosure.

In a hardware implementation, processing units used to perform the techniques may be implemented in one or more ASICs, DSPs, digital signal processing devices (DSPDs), programmable logic devices (PLDs), field programmable gate arrays (FPGAs), processors, controllers, microcontrollers, microprocessors, electronic devices, other electronic units designed to perform the functions described in the present disclosure, computer, or a combination thereof.

Accordingly, various example logic blocks, modules, and circuits described in connection with the present disclosure may be implemented or performed with general purpose processors, DSPs, ASICs, FPGAs or other programmable logic devices, discrete gate or transistor logic, discrete hardware components, or any combination of those designed to perform the functions described herein. The general purpose processor may be a microprocessor, but in the alternative, the processor may be any related processor, controller, microcontroller, or state machine. The processor may also be implemented as a combination of computing devices, for example, a DSP and microprocessor, a plurality of microprocessors, one or more microprocessors associated with a DSP core, or any other combination of the configurations.

In the implementation using firmware and/or software, the techniques may be implemented with instructions stored on a computer-readable medium, such as random access memory (RAM), read-only memory (ROM), non-volatile random access memory (NVRAM), programmable read-only memory (PROM), erasable programmable read-only memory (EPROM), electrically erasable PROM (EEPROM), flash memory, compact disc (CD), magnetic or optical data storage devices, etc. The commands may be executable by one or more processors, and may cause the processor(s) to perform certain aspects of the functions described in the present disclosure.

Although the embodiments described above have been described as utilizing aspects of the currently disclosed subject matter in one or more standalone computer systems, aspects are not limited thereto, and may be implemented in conjunction with any computing environment, such as a network or distributed computing environment. Furthermore, the aspects of the subject matter in the present disclosure may be implemented in multiple processing chips or apparatus, and storage may be similarly influenced across a plurality of apparatus. Such apparatus may include PCs, network servers, and portable apparatus.

Although the present disclosure has been described herein in connection with some embodiments, various modifications and changes can be made without departing from the scope of the present disclosure, which can be understood by those skilled in the art to which the present disclosure pertains. In addition, such modifications and changes should be considered within the scope of the claims appended herein.

### DESCRIPTION OF REFERENCE NUMERALS

110: User terminal
120: User
130: Dental image
140: Tooth information
150: Patient information
160: Guide data

1. A method for generating a guide for dental treatment, the method being executed by at least one processor and comprising:
receiving, from a user terminal, a position code for a tooth of a plurality of teeth assigned different position codes and a dental image including the tooth;
identifying a nerve region of the tooth in the received dental image, wherein the nerve region of the tooth includes the tooth and a nerve tissue around the tooth; and
outputting guide data for the tooth corresponding to the nerve region based on the position code and the identified nerve region.

2. The method according to claim 1, wherein the identifying the nerve region of the tooth in the received dental image includes:
selecting, from among a plurality of first machine learning models, a first machine learning model associated with the position code of the tooth; and
inputting the nerve region of the tooth to the selected first machine learning model to perform segmentation on the nerve region.

3. The method according to claim 2, wherein the outputting the guide data includes:
receiving, from the user terminal, a request to output first guide data;
selecting, from among a plurality of second machine learning models, a second machine learning model associated with the position code of the tooth; and
inputting the segmentation performed on the nerve region to the selected second machine learning model to output the first guide data.

4. The method according to claim 3, wherein
the first guide data includes at least one of a distance between a root tip and a bone level in the nerve region, a thickness and a length of a periodontal ligament in the nerve region, or an amount of bone in the nerve region, and
the first guide data is overlaid on the dental image and output, and is guide data for implant treatment.

5. The method according to claim 4, wherein the outputting the guide data includes:
receiving, from the user terminal, a request to output second guide data;
selecting, from among a plurality of third machine learning models, a third machine learning model associated with the position code of the tooth; and
inputting the segmentation performed on the nerve region to the selected third machine learning model to output the second guide data.

6. The method according to claim 5, wherein
the second guide data includes at least one of prep guide data, coordinates of a neural canal opening in the tooth, a number of neural canals, a length of a neural canal, or a shape of the neural canal, and
the second guide data is overlaid on the dental image and output, and is guide data for nerve treatment.

7. The method according to claim 6, further comprising:
classifying the plurality of teeth assigned the different position codes into at least one structure of central incisor, lateral incisor, canine, bicuspid, and molar;
training the plurality of first machine learning models to perform the segmentation on the nerve region in accordance with the classified tooth structure;
training the plurality of second machine learning models to output first guide data in accordance with the classified tooth structure; and
training the plurality of third machine learning models to output second guide data in accordance with the classified tooth structure.

8. The method according to claim 1, wherein the identifying the nerve region of the tooth includes:
detecting a plurality of tooth regions from the dental image; and
identifying the nerve region of the tooth based on the detected plurality of tooth regions and the received position code for the at least one tooth.

9. A non-transitory computer-readable recording medium storing a computer program that cause performance of the method according to claim 1 on a computer.

10. An information processing system, comprising:
a memory; and
at least one processor connected to the memory and configured to execute at least one computer-readable program included in the memory, wherein
the at least one program includes instructions for:
receiving, from a user terminal, a position code for at least one tooth of a plurality of teeth assigned different position codes and a dental image including the tooth;
identifying a nerve region of the tooth in the received dental image, wherein the nerve region of the tooth includes the tooth and a nerve tissue around the tooth; and
outputting guide data for the tooth corresponding to the nerve region based on the position code and the identified nerve region.
